(19) United States
(12) Patent Application Publication
(10) Pub. No.: US 2014/0271816 A1
(43) Pub. Date: Sep. 18, 2014

(54) TREATMENT OF POTENTIAL PLATELET AGGREGATION WITH LIPOSOMALLY FORMULATED GLUTATHIONE AND CLOPIDOGREL

(71) Applicant: Frederick Timothy Guilford, Palo Alto, CA (US)

(72) Inventor: Frederick Timothy Guilford, Palo Alto, CA (US)

(21) Appl. No.: 13/838,030

(22) Filed: Mar. 15, 2013

(51) Int. Cl. A61K 9/127; A61K 31/4365; A61K 31/59; A61K 31/445

(52) U.S. Cl. CPC A61K 9/127; A61K 31/445; A61K 31/4365; A61K 31/59
USPC 424/450; 514/327; 514/301; 514/167

(57) ABSTRACT

The composition of the invention, liposomal glutathione in combination with clopidogrel has utility for improving the efficacy of clopidogrel in preventing the aggregation of platelet that can lead to clotting. The prevention of platelet aggregation has widespread utilization in many cardiovascular conditions such as coronary artery narrowing and more consistent “antiplatelet” activity is found with the invention, the combination of clopidogrel and liposomal reduced glutathione.

### TECHNICAL FIELD

### Statement of Industrial Applicability

The invention relates to the use of liposomally formulated reduced glutathione to improve the antiplatelet aggregation therapy efficacy of clopidogrel in order to attenuate potential side effects of clopidogrel which has sufficiently severe and sufficiently common side effects to have the FDA mandate a so-called Black Box warning on the package insert (sold as Plavix® (registered trademark of Bristol-Myers-Squibb of 345 Park Ave., New York, N.Y.) and also sold by generic makers).

### BACKGROUND

Clopidogrel is an oral, thienopyridine class antiplatelet aggregation agent used to inhibit blood clots in coronary artery disease, peripheral vascular disease, and cerebrovascular disease. The goal of preventing platelet aggregation also known as “antiplatelet” activity is to prevent the formation of blood clots, particularly in individuals who undergo percutaneous coronary intervention and antiplatelet therapy with clopidogrel is in widespread use.

It has been observed that clopidogrel has significant variability in its efficacy between individuals. Nearly one-third of patients do not respond to clopidogrel therapy which has important clinical implications and may lead to recurrent cardiovascular events (Matetzky, Shenkman et al. 2004) (Mason, Freedman et al. 2004) It was initially thought that a polymorphism in the gene which codes for the protein Cytochrome P450 2C19 (abbreviated CYP2C19) and encoded by the CYP2C19 gene was the cause of the diminished response to clopidogrel therapy, The polymorphism known as CYP2C19*2 does not occur as frequently as the decreased response to treatment with clopidogrel and it appears that other factors are involved in the decreased effectiveness of clopidogrel.

The variable response to clopidogrel therapy is closely related to the observation that clopidogrel is a prodrug that requires activation by Cytochromes P450 (P450s) in order to form its pharmacologically active metabolite (AM). Increasing evidence supports the concept that clopidogrel's first oxidative metabolite is converted by P450s to the AM via a sulfinic intermediate. The highly unstable sulfenic acid is then rapidly reduced by reduced glutathione (GSH) to form a mixed disulfide conjugate (RS-SG) that is subsequently further reduced by another GSH molecule to form the active metabolite (AM). It is widely accepted that the AM is responsible for inhibition of platelet aggregation through covalent modification of the platelet P2Y₁₂ receptor. The activity of clopidogrel appears to rely on the interaction of GSH and the metabolites of the clopidogrel (Zhang, Lauver et al. 2013). The drug works by irreversibly inhibiting a receptor called P2Y12, an adenosine diphosphate (ADP) chemoreceptor on platelet cell membranes. The active metabolite has an elimination half-life of about eight hours and acts by forming a disulfide bridge with the platelet ADP receptor.

### SUMMARY OF INVENTION

### Technical Problem

In March 2010, the U.S. Food and Drug Administration (FDA) added a boxed warning to Plavix (the brand name of clopidogrel) (also applicable to prasugrel) (Members, Holmes et al. 2010) alerting that the drug can be less effective in people who cannot metabolize the drug to convert it to its active form (Matetzky, Shenkman et al. 2004). Recent research suggests that GSH is needed to facilitate the formation of the AM. It has been shown that GSH is the best reducing agent needed to form the active metabolites of clopidogrel in ex vivo studies. The technical problem is that plain GSH given orally to humans is not absorbed and has little tissue activity. Studies with a precursor of GSH, N-Acetyl cysteine (NAC) show that NAC has less than 10% of the activity of GSH in forming the AM.

The problem with administering glutathione is that the plain, non-formulated form of glutathione is not absorbed well after oral administration (Witschi, Reddy et al. 1992). The lack of absorption and function after oral administration is illustrated in a study by Levitskaia, in which plain GSH given orally has no function in removing a radiotagged marker (Co-60) from the liver, while IV GSH is able to remove 64% of the marker Levitskaia et al, Aminothiol Receptors for Decorporation of Intravenously Administered ⁶⁰Co In The Rat, Health Physics, Vol. 98(1) No. 4: 53-60 (Levitskaia, Morris et al. 2010).

### Solution to Problem

Based on research with a preparation of liposomally encapsulated reduced glutathione designed for oral use, the administration of the liposomal reduced glutathione (LRG) can provide systemic and intracellular glutathione to facilitate the production of the AM of clopidogrel.

The inventor also believes support with Vitamin D is important to support the efficacy of the GSH.

### Advantageous Effects of Invention

The advantage of LRG (liposomal reduced glutathione) is that it can be provided either orally or intravenously to provide reduced GSH to facilitate the formation of the active metabolites of clopidogrel. The combination of clopidogrel and LRG can be given acutely either orally or intravenously to prevent platelet aggregation during percutaneous coronary interventions and the same combination can be continued as an oral preparation to prevent platelet aggregation related clotting in the post-operative period for as long as is needed.

It has been shown that many individuals with coronary artery disease have low glutathione (De Chiara, Mafrici et al. 2007) (Ashfaq, Abramson et al. 2006), which may predispose to the difficulty in metabolizing clopidogrel. The administration of the combination of LRG in combination with clopidogrel will facilitate the rapid and continuous formation of the AM of clopidogrel.

### DESCRIPTION OF EMBODIMENTS

Research has shown that the use of N-Acetyl Cysteine (NAC), a building block of glutathione can have the effect of reversing the oxidative stress in cells. A lack of adequate glutathione in the defensive immune cells such as macrophages can lead to serious deficits in immune defense against infection as related in the discussion of the Venketaraman studies below. However as explained below, NAC requires the function of the enzymes needed to combine the three amino acids of glutathione as well as energy to formulate intracellular glutathione, A compromise of energy and enzyme function can occur because of oxidative stress induced by infection and related inflammatory problems. The lack of production of glutathione can lead to problems such as atherosclerosis with narrowing of arterial blood vessels as discussed in (Rosenblat, Volkova et al. 2007). Cysteine, as found in NAC has been the only possible oral method, however inefficient, to increase glutathione though it is not particularly effective (Levitskaia, Morris et al. 2010) (Witschi, Reddy et al. 1992). Non-formulated glutathione itself, as a tripeptide, does not survive passage of the gut to be physiologically effective to individual cells including heart tissue. Liposomally encapsulated reduced glutathione, the present invention, has been shown in a published study (Lauver, Kaissarian et al. 2012) to raise glutathione levels in tissues such as heart, liver and brain after oral ingestion in a rabbit model of ischemia (low oxygen) followed by the return of blood flow and oxygen (i.e., reperfusion) injury.

Research was commissioned at the University of Michigan, showing the surprising effect of the invention in reversing and controlling the oxidative stress and low glutathione in tissues such as that which occurs in individuals with severe illnesses. Individuals in the Intensive Care Unit (ICU) or undergoing coronary interventions, have been shown to be deficient in glutathione due to compromise of the enzymes responsible for the production of glutathione (Hammarqvist, Luo et al. 1997) (De Chiara, Mafrici et al. 2007). As noted in Lauver 2012, liposomal reduced glutathione can raise tissue levels of glutathione after oral ingestion. No publications reference the use of liposomal reduced glutathione to raise tissue levels of glutathione as documented in Lauver et al, University of Michigan Medical School, “Oral Pretreatment With Liposomal Glutathione Attenuates Reperfusion Injury in Rabbit Isolated Hearts,” (Lauver, Kaissarian et al. 2012). That study shows that contrary to the usual degradation in the gut, the invention, purchased from Your Energy Systems, LLC of Palo Alto, Calif., in the amount of approximately 428.8 mg of GSH administered in 5 ml doses, had the following abstracted result:
“A liposomal preparation of glutathione (lipGSH) capable of oral administration was investigated for its ability to attenuate tissue injury and increase myocardial glutathione levels in an isolated heart model of reperfusion injury. Male, New Zealand white rabbits were assigned randomly among four groups: control and daily oral administration of lipGSH for three, seven or fourteen days. At completion of the dosing regimen, hearts were harvested and perfused in a retrograde manner with the use of a Langendorff apparatus. The hearts were subjected to 30 min of global ischemia followed by 60 min of reperfusion. Hearts from lipGSH-treated rabbits exhibited better recovery of left ventricular contractile function during reperfusion and had attenuated oxidative damage. Furthermore, hearts from lipGSH-treated animals had increased myocardial tissue levels of GSH demonstrating effective absorption of lipGSH.”

The invention proposes that based on the Lauver et al research, the administration of liposomally encapsulated glutathione pursuant to the invention would raise the level of intracellular glutathione by at least 30%, particularly in tissues oxidatively stressed.

Plain, non-formulated glutathione used orally is not an option for this therapy as plain glutathione is not absorbed after oral ingestion in humans (Witschi, Reddy et al. 1992). A rat study of the removal of a radio-tagged metal (CO-60) from the liver, performed at Pacific Northwest National Laboratory with oral liposomally encapsulated reduced glutathione confirms this observation. The animals receiving:
a. Control (water only) showed 100% of the toxin remained=0% removal
b. Plain glutathione, oral, in water showed 100% of the toxin remained=0% removal.
c. Intravenous glutathione showed 36% of the toxin remaining=64% removal.
d. Liposomal reduced glutathione showed 53% of the toxin remaining=47% removal.

The data from this study is consistent with the observation that liposomally encapsulated glutathione is almost as effective as intravenous glutathione in removing the toxin. The plain glutathione has little if any absorption or efficacy. Levitskaia et al, Aminothiol Receptors for Decorporation of Intravenously Administered ⁶⁰Co In The Rat, Health Physics, Vol. 98(1) No. 4: 53-60 (Levitskaia, Morris et al. 2010).

The combination of oral liposomal glutathione plus clopidogrel can be administered as two individual preparations as the liposomal glutathione is a liquid and the clopidogrel is administered as a pill. The components of this invention can be administered separately or combined in a single capsule or dose.

Oral liposomally encapsulated reduced glutathione that is uniquely designed to be absorbed a) across the mucosa of the nose, mouth, gastrointestinal tract, b) after topical application for transdermal, or c) by intravenous infusion of glutathione with or without liposome encapsulation is prepared under the method and according to the composition described as follows:

### Basic Dosing Information

For a typical adult ranging from 55 kg to 90 kg, the dose of oral liposomally encapsulated reduced glutathione is 422 mg (1 teaspoon) (5 ml each) at least twice a day. More preferable is administration of 4 teaspoons (5 ml each) 4 times per day. If the initial does is tolerated well, a loading dose of another 1-5 teaspoons (5 ml-25 ml) after perhaps an hour would be helpful.

The concentration of the glutathione in the liposomes can be in a range from 3.3% w/w to 9% w/w or higher. The amount of 3.3% w/w corresponds to 123 mM concentration. Deionized water can be used to bring w/w percentages up to 100% w/w in any of the tables or formulations below.

### Dosing

Selenium should also be administered 200 microgram (μg) per day if there is inadequate selenium in a patient.

Liposomally encapsulated reduced glutathione (also referred to as liposomal glutathione or liposomal reduced glutathione or liposome-encapsulated glutathione): The preferred dosing schedule of the invention in combination with clopidogrel 75 mg is 800 mg (2 teaspoons) of the invention to be taken twice a day on an empty stomach (that is: do not ingest until 30 minutes after eating solid food) and may be administered orally or through a nasogastric tube. Clopidogrel is rapidly absorbed after oral administration of doses of 75 mg clopidogrel (base), with peak plasma levels (approx. 3 mg/L) of the main circulating metabolite occurring approximately one hour after dosing, So administering the LRG 800 mg simultaneously with 75 mg clopidogrel is convenient for achieving absorption and function of the combination. For purposes of this invention, whenever a reference is made to clopidogrel, it shall also apply to prasugrel, marketed as Effient® by Lilly USA LLC of Indianapolis, Ind. The base dose of 10 mg of prasugrel shall correspond to 75 mg clopidogrel, and dosages of prasugrel can be adjusted to correspond to the recommendations by taking the ratio of the recommended dose of clopidogrel in the various embodiments of this invention, and this base dose of clopidogrel of 75 mg and adjusting the dose of prasugrel by that ratio.

1 teaspoon (5 ml.) of the invention of oral liposomally encapsulated reduced glutathione contains approximately 420 mg reduced glutathione (“GSH”), and may contain 423 mg reduced glutathione, and 428 mg reduced glutathione. Liposomally encapsulated reduced glutathione is also referred to as liposomal reduced glutathione or LRG. References hereafter to GSH in association with quantities are meant to refer to liposomal reduced glutathione.

A preferred mode sets a suggested dose based on body weight. Gently stir liposomally encapsulated reduced glutathione into the liquid of your choice and take with clopidogrel in doses recommended.

Recommended dosing is 2 teaspoons of liposomal glutathione for every 75 mg clopidogrel administered.

### Determine Individual Dose by Body Weight: For Children

Under 30 lbs: ¼-½ teaspoon (1.25 ml-2.5 ml)=100-200 mg GSH
30-60 lbs: ½-1 teaspoon (2.5 ml-5 ml)=210-420 mg GSH
60-90 lbs: ¾-1.5 teaspoon (3.75 ml-5 ml)=316 mg-630 GSH
90-120 lbs: 1-2 teaspoon (5 ml-10 ml)=422-844 mg GSH
120-150 lbs: 1½-3 teaspoon (15 ml)=630-1260 mg GSH
Over 150 lbs: 1½-3 teaspoons (15 ml)=630-1260 mg GSH

The liposomal reduced glutathione should be used on a continuous basis once a patient is identified to be at risk of platelet aggregation or when a patient is prescribed clopidogrel. Children—should use a dose of liposomally encapsulated reduced glutathione equivalent to 60 mg/Kg of body weight daily in divided doses.

These doses should be continued for the duration of the illness and for purposes of maintaining adequate glutathione in tissues before, during and after clopidogrel therapy.

For maintaining the effect of the dosing schedules to follow, a second dose of LRG 800 mg should be taken for a twice day dosing schedule with the LRG to be taken on an empty stomach.

### Usual Adult Dose for Ischemic Stroke

75 mg clopidogrel+800 mg LRG mg orally once a day with or without food.

Aspirin therapy is optional in combination with clopidogrel and liposomal reduced glutathione, the present invention.

### Usual Adult Dose for Myocardial Infarction

75 mg clopidogrel+800 mg LRG orally once a day with or without food.

### Usual Adult Dose for Acute Coronary Syndrome—Prophylaxis

75 mg clopidogrel+800 mg LRG orally once a day with or without food.

### Usual Adult Dose for Peripheral Arterial Disease

75 mg clopidogrel+800 mg LRG orally once a day, with the dose taken on an empty stomach.

### Usual Adult Dose for Acute Coronary Syndrome

Unstable angina, non ST segment elevation myocardial infarction Initial: 300 mg of clopidogrel loading dose in combination with 5 teaspoons

(40 ml) of LRG, followed by 75 mg clopidogrel+800 mg LRG once daily for at least 1 month and ideally up to 12 months and optionally, (in combination with aspirin), clopidogrel 75 mg+800 mg LRG to 150 mg clopidogrel+1600 mg LRG once daily indefinitely). ST segment elevation acute myocardial infarction (STEMI): 75 mg clopidogrel+800 mg LRG once daily (in combination with aspirin 162 to 325 mg initially, followed by 81 to 162 mg/day);

### Usual Adult Dose for Percutaneous Coronary Intervention (PCI)

Percutaneous coronary intervention (PCI) for UA/NSTEMI or STEMI:

Loading dose: 300 to 600 mg clopidogrel (600 mg may be preferred for early invasive strategy with UA/NSTEMI) in combination with 40 ml ((3200 mg) to 80 ml (6400 mg) of LRG given as early as possible before or at the time of PCI followed by 75 mg+800 mg LRG once daily.

Higher versus standard maintenance dosing: A reasonably skilled practitioner in the art may also prefer a mode of a maintenance dose of 150 mg clopidogrel+1800 mg LRG once daily for 6 days, then clopidogrel 75 mg+800 mg LRG once daily thereafter in patients not at high risk for bleeding;

### Usual Pediatric Dose for Platelet Aggregation Inhibition

Note: Safety and efficacy have not been established in pediatric patients; optimal dose is not known; limited dosing information is available; further pediatric studies are needed.

Neonates and Infants up to 2 years: 0.2 mg/kg clopidogrel+6 mg/kg LRG once daily to achieve a mean inhibition of platelet aggregation similar to adults receiving the recommended dose.

Children over 2 years of age: Optimal dose is not established; some centers may use the following: Initial dose: 1 mg/kg clopidogrel+6 mg/kg of LRG once daily; titrate to response; in general, do not exceed adult dose.

The combination of clopidogrel and LRG described as the present invention can be used in the treatment of pediatric patients with a systemic to pulmonary artery shunt, intracardiac or intravascular stent, Kawasaki disease, or arterial graft.

### Reduced Dose Clopidogrel Plus Liposomal Reduced Glutathione for Patients at Risk of Bleeding

For patients at risk of bleeding who need anti-aggregation/anti-clotting support, the recommended dose is a ⅓ dose of 75 mg clopidogrel and 800 mg of liposomal reduced glutathione once a day or evenly distributed over multiple doses in a day.

### Precautions

Clopidogrel+LRG is contraindicated in patients with active pathological bleeding, such as peptic ulcer or intracranial hemorrhage and should be used cautiously in patients at increased risk of bleeding.

Caution is advised in patients receiving clopidogrel+LRG, particularly those who are also receiving aspirin, who require any type of parenteral access procedure (i.e., venipuncture, lumbar puncture, surgery). These patients are at a higher risk of hemorrhagic complications.

In cases where concern about excess anticoagulation or bleeding may be present, the following strategy of administration of clopidogrel or metabolites of clopidogrel in combination with oral liposomal glutathione for monitoring platelet aggregation may be used. An initial baseline reading of platelet aggregation is obtained using standard platelet aggregation testing using the test method for an aggregometer reading of the patient's blood. The initial dose of clopidogrel 75 mg in combination with oral liposomal glutathione 2 teaspoons is given. 12 to 24 hours later a test of platelet aggregation is obtained and is used to inform the subsequent dose. For example, if excessive aggregation of platelets is found, an increased dose of the combination of clopidogrel and oral liposomal glutathione may be given. If excessive bleeding or a rapid inhibition of platelet aggregation has been found the administration of oral liposomal glutathione may be continued without the administration of clopidogrel. Repeated testing of platelet aggregation can be used to inform the need for subsequent doses of clopidogrel.

Methods of platelet monitoring can be seen in U.S. Publication 20070065497 published Mar. 22, 2007 of an invention by Frederick Timothy Guilford entitled “Combination and method using EDTA combined with glutathione in the reduced state encapsulated in a liposome to facilitate the method of delivery of the combination as an oral, topical, intraoral or transmucosal, for anti-thrombin effect and for anti-platelet aggregation and measurement of efficacy.”

There also can be additional combination with the administration of Vitamin D3 or synthetic analogue of Vitamin D3 or vitamin D2 intravenously, intramuscularly, or orally in doses from 5000 IU to 100,000 IU or higher. This combination allows increased function of the enzyme glutathione reductase to regenerate glutathione that has been “used” as an antioxidant and is then in the form of oxidized glutathione (abbreviated “GSSG”) back into reduced glutathione abbreviated GSH.

The Vitamin D (25OH) range of blood levels is 30-100 ng/ml.

### DETAILED DESCRIPTION OF THE PREFERRED EMBODIMENTS

The methods of manufacture described in Keller et al U.S. Pat. No. 5,891,465, U.S. Pat. No. 6,610,322, and U.S. Pat. No. 6,726,924 and U.S. provisional application No. 60/597,041 by this inventor are adopted herein and into the modes of this invention and can be applied to the examples without undue experimentation. Liposomal formulations preferred in this invention can be purchased from Biozone, Inc. of Pittsburgh, Calif. Reduced glutathione can be purchased from Sigma-Aldrich of St. Louis, Mo. or from Kyowa Hakko USA, Inc., 767 3^{rd} Ave. No. 9, of New York City, New York 10017 with a Western regional office at 85 Enterprise, Suite 430, Aliso Viejo, Calif. 92656. Liposomally encapsulated reduced glutathione can be purchased from Your Energy Systems, LLC, 555 Bryant St., Suite 305, Palo Alto, Calif. 94301.

### Example 1

### Liposomal Glutathione Drink or Spray 2500 Mg Per Ounce or Form Suitable for Encapsulation or Gel

| | % w/w |
|---|---|
| Deionized Water | 74.4 |
| Glycerin | 15.00 |
| Lecithin | 1.50 |
| Potassium Sorbate | 0.10 |
| (optional spoilage retardant) | |
| Glutathione (reduced) | 8.25 |

A lipid mixture having components lecithin, and glycerin were commingled in a large volume flask and set aside for compounding. Hydroxylated lecithin is the preferred ingredient.

In a separate beaker, a water mixture having water, glycerin, glutathione were mixed and heated to, but not more than, 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of liposomally encapsulated glutathione. The amount of glutathione added to the formulation may range from 3.3% w/w to 8.5% w/w or higher. The methods of manufacture described in Keller et al U.S. Pat. No. 5,891,465, U.S. Pat. No. 6,958,160 and U.S. Pat. No. 7,150,883 and U.S. provisional application No. 60/597,041 are incorporated in this description. Concentrations of liposomally encapsulated glutathione from 3.3% w/w, 4% w/w, 5% w/w, 6% w/w, 7% w/w, 7.5% w/w, 8% w/w, 8.5% w/w or 9% w/w liposomally encapsulated glutathione may be formed and utilized for dosing by decreasing the amounts of glutathione and preplacing the material with an increase in the sterile water concentration.

### Example 1A

### Liposomally Encapsulated Reduced Glutathione Drink or Spray 2500 Mg Per Ounce or Form Suitable for Encapsulation or Gel: In %, According to w/w: Deionized Water 75, Glycerin 15.00, Lecithin 1.50, Extract Potassium Sorbate 0.10, Glutathione 8.5 (Reduced)

A lipid mixture having components lecithin, ethyl alcohol and glycerin were commingled in a large volume flask and set aside for compounding. Hydroxylated lecithin is the preferred ingredient.

In a separate beaker, a water mixture having water, glycerin, glutathione were mixed and heated, but not more than, 50.degree C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. A spoilage retardant such as potassium sorbate or BHT would be added. The solution would be placed in appropriate dispenser for ingestion as a liquid or administration as a spray. Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of liposomally encapsulated glutathione. The amount of glutathione added to the formulation may range from 3.3% w/w to 8.5% w/w or higher. The methods of manufacture described in Keller et al U.S. Pat. No. 5,891,465, U.S. Pat. No. 6,958,160 and U.S. Pat. No. 7,150,883 and U.S. provisional application No. 60/597,041 are incorporated in this description.

Concentrations of liposomally encapsulated glutathione from 3.3% w/w, 4% w/w, 5% w/w, 6% w/w, 7% w/w, 7.5% w/w, 8% w/w, 8.5% w/w or 9% w/w liposomally encapsulated glutathione may be formed and utilized for dosing by decreasing the amounts of glutathione and preplacing the material with an increase in the sterile water concentration.

### Example 2

Embodiment two of the invention includes the incorporation of the fluid liposome (such as that prepared in Example 1A) into a gelatin based capsule to improve the stability, provide a convenient dosage form, and assist in sustained release characteristics of the liposome. The present embodiment relates to the use of glutathione in the reduced state encapsulated into liposomes or formulated as a preliposome formulation and then put into a capsule. The capsule can be a soft gel capsule capable of tolerating a certain amount of water, a two-piece capsule capable of tolerating a certain amount of water or a two-piece capsule where the liposomes are preformed then dehydrated.

The liposome-capsule unit containing biologically encapsulated material can be taken in addition to orally, used for topical unit-of-use application, or other routes of application such as intra-ocular, intranasal, rectal, or vaginal.

The composition of examples 1 and 2 may be utilized in the encapsulated embodiment of this invention.

Gelatin capsules have a lower tolerance to water on their interior and exterior. The usual water tolerance for a soft gel capsule is 10% w/w on the interior. The concentration of water in a liposome formulation can range from 60-90% water. An essential component of the present invention is the formulation of a liposome with a relatively small amount of water, in the range of 5-10% w/w. By making the liposome in a low aqueous system, the liposome is able to encapsulate the biologically active material and the exposure of water to the inside lining of the capsule is limited. The concentration of water should not exceed that of the tolerance of the capsule for which it is intended. The preferred capsule for this invention is one that can tolerate water in the 15-20% w/w range.

The methods described by Keller et al, U.S. Pat. No. 6,726,924 are incorporated in this description.

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. Filling of gel caps on a large scale is best with the rotary die method or others such as the Norton capsule machine.

### Example 3

Embodiment number four of the present invention includes the creation of liposome suspension using a self-forming, thermodynamically stable liposomes formed upon the adding of a diacylglycerol-PEG lipid to an aqueous solution when the lipid has appropriate packing parameters and the adding occurs above the melting temperature of the lipid. The method described by Keller et al, U.S. Pat. No. 6,610,322 is incorporated into this description.

Most, if not all, known liposome suspensions are not thermodynamically stable. Instead, the liposomes in known suspensions are kinetically trapped into higher energy states by the energy used in their formation. Energy may be provided as heat, sonication, extrusion, or homogenization. Since every high-energy state tries to lower its free energy, known liposome formulations experience problems with aggregation, fusion, sedimentation and leakage of liposome associated material. A thermodynamically stable liposome formulation which could avoid some of these problems is therefore desirable.

The present embodiment prefers liposome suspensions which are thermodynamically stable at the temperature of formation. The formulation of such suspensions is achieved by employing a composition of lipids having several fundamental properties. First, the lipid composition must have packing parameters which allow the formation of liposomes. Second, as part of the head group, the lipid should include polyethyleneglycol (PEG) or any polymer of similar properties which sterically stabilizes the liposomes in suspension. Third, the lipid must have a melting temperature which allows it to be in liquid form when mixed with an aqueous solution.

By employing lipid compositions having the desired fundamental properties, little or no energy need be added when mixing the lipid and an aqueous solution to form liposomes. When mixed with water, the lipid molecules disperse and self assemble as the system settles into its natural low free energy state. Depending on the lipids used, the lowest free energy state may include small unilamellar vesicle (SUV) liposomes, multilamellar vesicle (MLV) liposomes, or a combination of SUVs and MLVs.

In one aspect, the invention includes a method of preparing liposomes. The method comprises providing an aqueous solution; providing a lipid solution, where the solution has a packing parameter measurement of Pₐ (Pₐ. references the surface packing parameter) between about 0.84 and 0.88, a Pᵥ (Pᵥ references the volume packing parameter) between about 0.88 and 0.93, (See, D. D. Lasic, Liposomes, From Physics to Applications, Elsevier, p. 51 1993), and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolylglycerol-PEG-12, either alone or as one of the lipids in a mixture. The method may further comprise providing an active compound, in this case glutathione (reduced); and combining the active compound with the lipid solution and the aqueous solution.

The low molecular weight in the preferred embodiments more effectively deliver the liposomally encapsulated reduced glutathione in active reduced form as needed and thus result in the surprising effect of the invention. The absorption into cells is a particular advantage of the preferred embodiment of the invention.

### Further Examples 4

### Formulation for Topical Application of Liposomally Encapsulated Reduced Glutathione

A topical cream or lotion containing reduced glutathione in a self-forming liposome sold under the brand name “QuSome” ® by Biozone Laboratories, Inc. of Pittsburgh, Calif. is another preferred embodiment. The Qusome self-forming liposome can be formed containing reduced liposomally encapsulated glutathione in a concentration of 5% reduced glutathione encapsulated in the liposome. Most liposomes use energy provided as heat, sonication, extrusion, or homogenization for their formation, which gives them a high energy state. Some liposome formulations can experience problems with aggregation, fusion, sedimentation and leakage of liposome associated material which this invention seeks to minimize and does minimize. The Qusome is a more thermodynamically stable liposome formulation. The Qusome self-forming liposome is self-forming at room temperature which that the mixing of the lipid and an aqueous lipid containing solution avoids alteration of the contents by heating. The resulting liposome is in a low free energy state so it remains stable and reproducible. The formulation of this embodiment is reviewed in example 3. The methods of manufacture described in Keller et al U.S. U.S. Pat. No. 6,958,160 and U.S. Pat. No. 7,150,883 are incorporated in this description. The most important details of that manufacturing are as follows:

The lipids used to form the lipid vesicles and liposomes in the present formulations can be naturally occurring lipids, synthetically made lipids or lipids that are semisynthetic. Any of the art known lipid or lipid like substances can be used to generate the compositions of the present invention. These include, but are not limited to, lecithin, ceramides, phosphatidylethanolamine, phosphotidylcholine, phosphatidylserine, cardiolipin and the like. Such lipid components for the preparation of lipid vesicles are well known in the art, for example see U.S. Pat. No. 4,485,954, and “Liposome Technology”, 2nd Ed, Vol. I (1993) G. Gregoriadis ed., CRC Press, Boca Raton, Fla.

Lipids with these properties that are particularly preferred in the present formulations include phospholipids, particularly highly purified, unhydrogenated lecithin containing high concentrations of phosphotidylcholine, such as that available under the trade name Phospholipon 90 from American Lecithin, or Nattermann Phospholipid, 33 Turner Road, Danbury, Conn. 06813-1908.

In formulating the liposomes, in one aspect, the invention includes a method of preparing liposomes. The method comprises providing an aqueous solution; providing a lipid solution, where the solution has a Pₐ between about 0.84 and 0.88, a Pᵥ between about 0.88 and 0.93, and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolyglycerol-PEG-12, either alone or as one of the lipids in a mixture. The method may further comprise providing an active compound; and combining the active compound with the lipid solution and the aqueous solution.

In another aspect, the invention includes a liposome suspension. The suspension comprises one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain. The PEG chain preferably has a molecular weight between about 300 Daltons and 5000 Daltons. The suspension may comprise a single lipid. The lipid may comprise dioleolylglycerol-PEG-12. The suspension may further comprise an active compound, which may be selected from the group described above.

In another aspect, the invention includes a composition for combining with an aqueous solution to form a liposome suspension. The composition comprises one or more lipids, where the lipids as an aggregate have a Pₐ between about 0.84 and 0.88, a Pᵥ, between about 0.88 and 0.93 and a melting temperature of between about 0 to 100 degrees centigrade; and where at least one lipid includes a polyethyleneglycol (PEG) chain. The PEG chain preferably has a molecular weight between about 300 Daltons and 5000 Daltons. The composition may comprise a single lipid. The composition may comprise dioleolylglycerol-PEG 12. The composition may further comprise an active compound selected from the group above. The composition may be provided in a sealed container, where the container also contains an inert gas to prevent oxidative degradation.

In another aspect, the invention includes a method of intravenously administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension intravenously. The method may further comprise providing kinetic energy to the liposome suspension. The method may also include providing the composition in a sealed container containing an inert gas. The PEG chain preferably has a molecular weight between about 300 Daltons and 5000 Daltons. The composition may comprise a single lipid. The lipid may comprise dioleolylglycerol-PEG-12. The active compound may be selected from the group above.

In another aspect, the invention includes a method of solubilizing an active compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing the active compound; providing an aqueous solution; and combining the active compound, the lipid and the aqueous solution to form a liposome suspension. The method may further comprise providing kinetic energy to the liposome suspension. The method may include providing the composition in a sealed container containing an inert gas. The PEG chain preferably has a molecular weight between about 300 Daltons and 5000 Daltons. The composition may comprise a single lipid. The lipid may comprise dioleolylglycerol-PEG-12. The active compound may be selected from the group above.

In another aspect, the invention includes a method of orally administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension orally in the form selected from the group comprising a two piece hard gelatin capsule, a soft gelatin capsule, or drops.

The compositions may be administered topically, inter-orally, vaginally or rectally.

PEG-12 Glyceryl Dioleate was obtained from Global 7 (New Jersey) for the following formulations. This can be substituted for the lecithin w/w % as needed to accomplish the formulation, or applied as set forth below.

In the following formulations, the “set percentage” w/w % of reduced glutathione is selected from 3.3%, 4%, 5%, 6%, 7%, 7.5%, 8%, 8.5% or 9% or amounts approximately to those percentages.

### Example 5A

### Spontaneous Liposomes for Intravenously Administering Therapeutic Compounds or for a Spray or Drink

A set percentage of reduced glutathione is dissolved in a sufficient amount of the solvent PEG-12 Glyceryl Dioleate, also called dioleolylglycerol-PEG 12, (either referred to as “PEGDO”) and gently mixed for about 5 minutes. A sufficient amount of PEGDO should be about 10% w/w. Deionized water is slowly added to the solution. Ingredients other than deionized water, the reduced glutathione and the PEGDO may be added such as preferably 0.1% w/w potassium sorbate and then the final amount of deionized water added is that amount which is necessary to have the percentages add up to 100% w/w. Taste or other flavor-masking ingredients could also be added before the deionized water is brought up to 100% w/w. Although taste ingredients can be added before or after the liposomal encapsulation formulation, the preferable mode is to add flavor or other taste masking ingredients after liposomal encapsulation formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10% w/w. The inventor has preferably used citrus seed extract.

### Example 5B

### Spontaneous Liposomes for Intravenously Administered Therapeutic Compound and as a Drug Solubilization Vehicle for Use in Spray or Drink

A set percentage of reduced glutathione is mixed with a sufficient amount of PEG-12 Glyceryl Dioleate, also called dioleolylglycerol-PEG 12, (either referred to as “PEGDO”) to bring the reduced glutathione into solution by vortexing and sonication for 10 minutes. A sufficient amount of PEGDO should be about 5% w/w. Deionized water is added and gently mixed. Ingredients other than deionized water, the reduced glutathione and the PEGDO may be added such as preferably 0.1% w/w potassium sorbate and then the final amount of deionized water added is that amount which is necessary to have the percentages add up to 100% w/w. Ingredients other than deionized water, the reduced glutathione and the PEGDO may be added such as preferably 0.1% w/w potassium sorbate and then the final amount of deionized water added is that amount which is necessary to have the percentages add up to 100% w/w. Taste ingredients or other flavor masking ingredients could also be added before the deionized water is brought up to 100% w/w. Although taste ingredients can be added before or after the liposomal formulation, the preferable mode is to add flavor or other taste masking ingredients after liposomal formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10% w/w. The inventor has preferably used citrus seed extract.

The QuSome self-forming liposome uses polyethyleneglycol (PEG) is a steric stabilizer and the resulting liposome is of a moderate size, 150 nm-250 nm. The combination of 150 nm-250 nm size and the PEG component is known to create long circulating liposomes. The size of the QuSome self-forming liposome allows them to be sterile filtered.

The concentration of liposomally encapsulated glutathione in the liposomes resulting from the Qusome formulation is 5% w/w for topical application. It is possible to use the Qusome technology in creating an oral formulation also and the 8.25% glutathione in w/w concentration encapsulated in the liposome may be used in the oral formulation.

### Further Examples and Embodiments

### Example 6

### GSNO Example

### Liposomally Encapsulated S-Nitroso-L-Glutathione (GSNO) Drink or Spray 2500 Mg Per Ounce or Form Suitable for Encapsulation or Gel

| | % w/w |
|---|---|
| Deionized Water | 74.4 |
| Glycerin | 15.00 |
| Lecithin | 1.50 |
| Potassium Sorbate | 0.10 |
| (optional spoilage retardant) | |
| GSNO | 8.25 |

Another method of supporting antiplatelet aggregation (preventing the aggregation of platelets) treatment is the liposomal encapsulation of GSNO to be taken at the same time as clopidogrel. GSNO (S-nitroso-L-glutathione) encapsulation in either the lecithin or the self forming liposomes of the current invention for use in combination with clopidogrel therapy. Liposomal encapsulated GSNO, molecular weight 336.3, is 80 mg/ml of liposomal GSNO and the dosing is ½ teaspoon (0.25 ml) in combination with clopidogrel 20 mg, to 4 teaspoons (20 ml.) orally twice a day in combination with clopidogrel 150 mg. This combination of doses of GSNO combined with, which means taken at the same time as clopidogrel whether encapsulated in the same capsule or taken orally at the same time as clopidogrel as both materials are rapidly absorbed after oral ingestion. The combinations of GSNO and clopidogrel gives ranges of doses includes ½ teaspoon (2.5 ml) combined with clopidogrel 20 mg, 1 teaspoon (5 ml) combined with clopidogrel 40 mg, and 2 teaspoon (10 ml) combined with clopidogrel 75 mg continuing these increments will give ranges from low dose up to routine dosing schedules. These dosing increments may be used once or in some cases twice a day.

In cases where concern about excess anticoagulation or bleeding may be present, the following strategy of administration of clopidogrel or metabolites of clopidogrel in combination with oral liposomal GSNO for monitoring platelet aggregation may be used. An initial baseline reading of platelet aggregation is obtained using standard platelet aggregation testing using the test method for an aggregometer reading of the patient's blood. The initial dose of clopidogrel 75 mg in combination with GSNO 2 teaspoons is given. 12 to 24 hours later a test of platelet aggregation is obtained and is used to inform the subsequent dose. For example, if excessive aggregation of platelets is found, an increased dose of the combination of clopidogrel and liposomal GSNO may be given. If excessive bleeding or a rapid inhibition of platelet aggregation has been found the administration of liposomal GSNO may be continued without the administration of clopidogrel. Repeated testing of platelet aggregation can be used to inform the need for subsequent doses of clopidogrel.

### REFERENCES

Ashfaq, S., J. L. Abramson, et al. (2006). “The relationship between plasma levels of oxidized and reduced thiols and early atherosclerosis in healthy adults.” J Am Coll Cardiol 47(5): 1005-1011.
De Chiara, B., A. Mafrici, et al. (2007). “Low plasma glutathione levels after reperfused acute myocardial infarction are associated with late cardiac events.” Coron Artery Dis 18(2): 77-82.
Hammarqvist, F., J. L. Luo, et al. (1997). “Skeletal muscle glutathione is depleted in critically ill patients.” Crit Care Med 25(1): 78-84.
Lauver, D. A., N. M. Kaissarian, et al. (2012). “Oral Pretreatment With Liposomal Glutathione Attenuates Reperfusion Injury in Rabbit Isolated Hearts.” Journal of cardiovascular pharmacology.
Levitskaia, T. G., J. E. Morris, et al. (2010). “Aminothiol receptors for decorporation of intravenously administered (60)Co in the rat.” Health Phys 98(1): 53-60.
Mason, P. J., J. E. Freedman, et al. (2004). “Aspirin resistance: current concepts.” Reviews in cardiovascular medicine 5(3): 156-163.
Matetzky, S., B. Shenkman, et al. (2004). “Clopidogrel resistance is associated with increased risk of recurrent atherothrombotic events in patients with acute myocardial infarction.” Circulation 109(25): 3171-3175.
Members, W. C., D. R. Holmes, et al. (2010). “ACCF/AHA Clopidogrel Clinical Alert: Approaches to the FDA “Boxed Warning”: A Report of the American College of Cardiology Foundation Task Force on Clinical Expert Consensus Documents and the American Heart Association.” Circulation 122(5): 537-557.
Rosenblat, M., N. Volkova, et al. (2007). “Anti-oxidant and anti-atherogenic properties of liposomal glutathione: studies in vitro, and in the atherosclerotic apolipoprotein E-deficient mice.” Atherosclerosis 195(2): e61-68.
Witschi, A., S. Reddy, et al. (1992). “The systemic availability of oral glutathione.” Eur J Clin Pharmacol 43(6): 667-669.
Zhang, H., D. A. Lauver, et al. (2013). “Formation, Reactivity, and Anti-platelet Activity Of Mixed Disulfide Conjugates Of Clopidogrel.” Molecular pharmacology.

1. A pharmaceutical composition for prevention of platelet aggregation comprising:
a compound selected from the group of anti-platelet aggregation drugs comprising clopidogrel and prasugrel; and
orally administering, to a patient having disease symptoms related to potential platelet aggregation, a dose of a reduced glutathione stabilized and encapsulated in a liposomal pharmaceutical carrier capable of being ingested orally, and capable of delivering glutathione (reduced) in a physiologically active state to improve said disease symptoms by transfer of the glutathione into animal cells, where the concentration of reduced glutathione in the entrapped aqueous space of the liposomes is at least 123 mM.

2. The composition of a combination of clopidogrel and reduced glutathione stabilized and encapsulated in a liposomal pharmaceutical carrier capable of being ingested orally, and capable of delivering glutathione (reduced) in a physiologically active state to decrease platelet aggregation where the concentration of reduced glutathione in the entrapped aqueous space of the liposomes is at least 123 mM for treatment to prevent platelet aggregation.

3. A method of treatment of patients having narrowing of arterial blood flow causing a propensity to ischemic stroke, or myocardial infarction, comprising the following steps:
administering a compound selected from the group of anti-platelet aggregation drugs comprising clopidogrel and prasugrel, and
administering reduced glutathione stabilized and encapsulated in a liposomal pharmaceutical carrier capable of being ingested orally, and capable of delivering glutathione (reduced) in a physiologically active state to decrease platelet aggregation where the concentration of reduced glutathione in the entrapped aqueous space of the liposomes is at least 123 mM.

4. A method of treatment of patients at risk of acute coronary syndrome (ACS), comprising the following steps:
administering a compound selected from the group of anti-platelet aggregation drugs comprising clopidogrel and prasugrel, and
administering reduced glutathione stabilized and encapsulated in a liposomal pharmaceutical carrier capable of being ingested orally, and capable of delivering glutathione (reduced) in a physiologically active state to decrease platelet aggregation where the concentration of reduced glutathione in the entrapped aqueous space of the liposomes is at least 123 mM.

5. A method of treatment of patients at risk of peripheral arterial disease, comprising the following steps:
administering a compound selected from the group of anti-platelet aggregation drugs comprising clopidogrel and prasugrel, and
administering reduced glutathione stabilized and encapsulated in a liposomal pharmaceutical carrier capable of being ingested orally, and capable of delivering glutathione (reduced) in a physiologically active state to decrease platelet aggregation where the concentration of reduced glutathione in the entrapped aqueous space of the liposomes is at least 123 mM.

6. A method of treatment of patients for percutaneous coronary artery intervention, comprising the following steps:
administering a compound selected from the group of anti-platelet aggregation drugs comprising clopidogrel and prasugrel, and
administering reduced glutathione stabilized and encapsulated in a liposomal pharmaceutical carrier capable of being ingested orally, and capable of delivering glutathione (reduced) in a physiologically active state to decrease platelet aggregation where the concentration of reduced glutathione in the entrapped aqueous space of the liposomes is at least 123 mM.

7. A method of treatment of pediatric patients at risk of platelet aggregation having a disease selected from the group of systemic to pulmonary artery shunt, intracardiac stent, intravascular stent, Kawasaki disease, or arterial graft, comprising the following steps:
administering 1 mg/kg clopidogrel; and
administering 6 mg/kg weight per day of reduced glutathione stabilized and encapsulated in a liposomal pharmaceutical carrier capable of being ingested orally, and capable of delivering glutathione (reduced) in a physiologically active state to decrease platelet aggregation where the concentration of reduced glutathione in the entrapped aqueous space of the liposomes is at least 123 mM.

8. A method of treatment of patients at risk of excess anti-coagulation or bleeding comprising the following steps:
reducing the base dose by two-thirds and then administering a compound selected from the group of anti-platelet aggregation drugs comprising clopidogrel and prasugrel; and
administering reduced glutathione stabilized and encapsulated in a liposomal pharmaceutical carrier capable of being ingested orally, and capable of delivering glutathione (reduced) in a physiologically active state to decrease platelet aggregation where the concentration of reduced glutathione in the entrapped aqueous space of the liposomes is at least 123 mM.

9. A pharmaceutical composition for prevention of platelet aggregation comprising:
a compound selected from the group of anti-platelet aggregation drugs comprising clopidogrel and prasugrel; and
liposomal GSNO.

10. The composition of a combination of clopidogrel and GSNO stabilized and encapsulated in a liposomal pharmaceutical carrier capable of being ingested orally for treatment to prevent platelet aggregation.

11. A method of treatment of patients having narrowing of arterial blood flow causing a propensity to ischemic stroke, or myocardial infarction, comprising the following steps:
administering a compound selected from the group of anti-platelet aggregation drugs comprising clopidogrel and prasugrel, and
administering GSNO stabilized and encapsulated in a liposomal pharmaceutical carrier capable of being ingested orally.

12. A method of treatment of patients at risk of excess anti-coagulation or bleeding comprising the following steps:
reducing the base dose by two-thirds and then administering a compound selected from the group of anti-platelet aggregation drugs comprising clopidogrel and prasugrel; and
administering reduced glutathione stabilized and encapsulated in a liposomal pharmaceutical carrier capable of being ingested orally, and capable of delivering glutathione (reduced) in a physiologically active state to decrease platelet aggregation where the concentration of reduced glutathione in the entrapped aqueous space of the liposomes is at least 123 mM.

13. The composition according to any one of claim 1, 2, 9 or 10, further comprising:
Vitamin D.

14. The method of treatment according to any one of claims 3 through 8, or 11 through 12, further comprising:
administering vitamin D.
